SEQUENCE LISTING (5 sequences)

SEQ ID NO: 1 - protein, 468 aa, Escherichia coli
SAEHVLTMLNEHEVKFVDLRFTDTKGKEQHVTIPAHQVNAEFFEEGKMFDGSSIGGWKGINESDMVLMPDASTAVIDPFFADSTLIIRCDILEPGTLQGYDRDPRSIAKRAEDYLRSTGIADTVLFGPEPEFFLFDDIRFGSSISGSHVAIDDIEGAWNSSTQYEGGNKGHRPAVKGGYFPVPPVDSAQDIRSEMCLVMEQMGLVVEAHHHEVATAGQNEVATRFNTMTKKADEIQIYKYVVHNVAHRFGKTATFMPKPMFGDNGSGMHCHMSLSKNGVNLFAGDKYAGLSEQALYYIGGVIKHAKAINALANPTTNSYKRLVPGYEAPVMLAYSARNRSASIRIPVVSSPKARRIEVRFPDPAANPYLCFAALLMAGLDGIKNKIHPGEAMDKNLYDLPPEEAKEIPQVAGSLEEALNELDLDREFLKAGGVFTDEAIDAYIALRREEDDRVRMTPHPVEFELYYSV

SEQ ID NO: 2 - DNA, 1410 bp, Escherichia coli
atgtccgctgaacacgtactgacgatgctgaacgagcacgaagtgaagtttgttgatttgcgcttcaccgatactaaaggtaaagaacagcacgtcactatccctgctcatcaggtgaatgctgaattcttcgaagaaggcaaaatgtttgacggctcctcgattggcggctggaaaggcattaacgagtccgacatggtgctgatgccagacgcatccaccgcagtgattgacccgttcttcgccgactccaccctgattatccgttgcgacatccttgaacctggcaccctgcaaggctatgaccgtgacccgcgctccattgcgaagcgcgccgaagattacctgcgttccactggcattgccgacaccgtactgttcgggccagaacctgaattcttcctgttcgatgacatccgtttcggatcatctatctccggttcccacgttgctatcgacgatatcgaaggcgcatggaactcctccacccaatacgaaggtggtaacaaaggtcaccgtccggcagtgaaaggcggttacttcccggttccaccggtagactcggctcaggatattcgttctgaaatgtgtctggtgatggaacagatgggtctggtggttgaagcccatcaccacgaagtagcgactgctggtcagaacgaagtggctacccgcttcaataccatgaccaaaaaagctgacgaaattcagatctacaaatatgttgtgcacaacgtagcgcaccgcttcggtaaaaccgcgacctttatgccaaaaccgatgttcggtgataacggctccggtatgcactgccacatgtctctgtctaaaaacggcgttaacctgttcgcaggcgacaaatacgcaggtctgtctgagcaggcgctgtactacattggcggcgtaatcaaacacgctaaagcgattaacgccctggcaaacccgaccaccaactcttataagcgtctggtcccgggctatgaagcaccggtaatgctggcttactctgcgcgtaaccgttctgcgtctatccgtattccggtggtttcttctccgaaagcacgtcgtatcgaagtacgtttcccggatccggcagctaacccgtacctgtgctttgctgccctgctgatggccggtcttgatggtatcaagaacaagatccatccgggcgaagccatggacaaaaacctgtatgacctgccgccagaagaagcgaaagagatcccacaggttgcaggctctctggaagaagcactgaacgaactggatctggaccgcgagttcctgaaagccggtggcgtgttcactgacgaagcaattgatgcgtacatcgctctgcgtcgcgaagaagatgaccgcgtgcgtatgactccgcatccggtagagtttgagctgtactacagcgtctaa

SEQ ID NO: 3 - DNA, 27 bp, Artificial Sequence
  synthetic DNA
attctagatttcgttaccacgacgacc

SEQ ID NO: 4 - DNA, 26 bp, Artificial Sequence
  synthetic DNA
ataagcttcacgttggagagcgactc

SEQ ID NO: 5 - DNA, 36 bp, Artificial Sequence
  synthetic DNA
gcgaagccatggacaaaaacctgtttgacctgccgc

(19) United States
(12) Patent Application Publication
(10) Pub. No.: US 2005/0255567 A1
(43) Pub. Date: Nov. 17, 2005

(54) Mutant glutamine synthetase and method for producing amino acids

(75) Inventors: Mikhail Markovich Gusyatiner, Moscow (RU); Lirina Valerievna Ivanovskaya, Moscow (RU); Tatyana Viktorovna Leonova, Moscow (RU); Ekaterina Igorevna Mukhanova, Moscow (RU); Yulia Georgievna Rostova, Moscow (RU); Dmitriy Vladimirovich Filippov, Moscow (RU); Daria Aleksandrovna Chudakova, Moscow (RU)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/167,273

(22) Filed: Jun. 28, 2005

Related U.S. Application Data
(62) Division of application No. 10/299,799, filed on Nov. 20, 2002.

(30) Foreign Application Priority Data
Nov. 30, 2001 (RU) 2001132473

(51) Int. Cl. C07H 21/04; C12P 13/14; C12N 9/10; C12N 1/21

(52) U.S. Cl. 435/110; 435/252.33; 435/193; 536/23.2

(57) ABSTRACT

Amino acids, such as L-glutamine, L-arginine, L-tryptophan, L-histidine and L-glutamate are produced using a bacterium belonging to the genus Escherichia harboring a mutant glutamine synthetase in which the tyrosine amino acid residue corresponding to position 397 in a wild type glutamine synthetase is replaced with any of amino acid residues, preferably with phenylalanine.

### BACKGROUND OF THE INVENTION

1. Field of the Invention

The present invention relates to microbiological industry, specifically to a method for producing amino acids. More specifically, the present invention concerns an use of a new enzyme involved in glutamine biosynthesis and nitrogen assimilation pathways of E. coli strains producing amino acids, such as glutamine and arginine. More specifically, the present invention concerns a new mutant glutamine synthetase and a method for producing amino acids, such as glutamine, arginine, tryptophan, histidine and glutamate, using E. coli strains harboring the enzyme.

2. Description of the Related Art

Glutamine synthetase (GS) has two functions in E. coli: the formation of glutamine and assimilation of ammonia when the availability of ammonia is restricted. Glutamine donates nitrogen for the synthesis of purines and pyrimidines, and for some amino acids, such as arginine, tryptophan, asparagine, histidine and glutamate. In case of arginine biosynthesis, glutamine plays significant role, since glutamine is used as the only physiological amino group donor for synthesis of carbamoylphosphate, which is a common precursor for arginine and the pyrimidines. In case of tryptophan formation, glutamine is utilized in the first reaction of tryptophan biosynthetic pathway, which involves the conversion of chorismate and glutamine to anthranilate, glutamate, and pyruvate. The glutamine-dependent asparagine synthetase uses glutamine together with asparatate and ATP in the major pathway for asparagine biosynthesis. The nitrogen 3 of imidazole ring of histidine originates from glutamine. And finally, glutamine is used by glutamate oxoglutarate aminotransferase (GOGAT) in the synthesis of glutamate.

Because of the multiple functions and importance of GS in cellular metabolism both its catalytic activities and its synthesis are highly regulated.

The overall structure of GS consists of 12 subunits arranged as two hexamers, face to face. Adenylylation of Tyr-397 of each subunit of GS down-regulates enzymatic activity in vivo. Both the adenylylation and de-adenylylation of GS are catalyzed by adenyltransferase, coded by the ginE gene. The direction of the catalysis is dictated by the regulatory proteine PII (glnB), the activity of which is also modulated by reversible modification: the unmodified form of PII activates the adenylylation, whereas the uridylylated form activates de-adenylylation of GS. A specific uridylyltransferase catalyzes transfer of a uridylyl group from UTP to PII, whereas uridylyl-removing activity reverses uridylytion of PII. Both activities are determined by the gene glnD. Glutamine stimulates urydylyl-removing activity, 2-oxoglutarate stimulates uridylytion of PII. So, eventually glutamine brings about adenylylation of GS, whereas 2-oxoglutarate promotes the formation of de-adenylylated (the active form) GS (Escherichia coli and Salmonela, Second Edition, Editor in Chief: F. C. Neidhardt, ASM Press, Washington D.C., 1996).

Earlier the mutant non-adenylylatable glutamine synthetases from different species have been described.

There are mutant GS from Rhizobium meliloti (Arcondeguy et al, FEMS Microbiol. Lett., 1996, 145:1, 33-40), Y398F mutant GS from Rhodospirillum rubrum (Zhang et al, J. Bacteriol., 2000, 182:4, 938-92) and Y407F mutant GS from Azobacter vinelandii (Colnaghi et al, Microbiology, 2001, 147:5, 1267-76). The cited mutated GS showed level of activity of the natural enzyme. But there are no reports of using the mutated GS lacking the ability to adenylylation for production of amino acids.

### SUMMARY OF THE INVENTION

The present invention is concerned with the construction of mutant and high active enzyme playing a key role in biosynthesis of glutamine and arginine in E. coli.

In the present invention the substitution of TAT codon, encoding tyrosine at position 397 in GS protein, by TTT codon, encoding phenylalanine amino acid residue, in glnA gene is proposed. The substitution of the amino acid residue in the amino acid sequence leads to expression of a mutant protein with native level of activity and free from adenylylation. It was found that the GS, mutated as above, became insensitive to indirect down-regulation by glutamine. Then the present inventors found that the glutamic acid producing bacterium belonging to the genus Escherichia transformed with a DNA harboring the mutant glnA gene become able to produce glutamine. Thus the present invention has been accomplished.

That is the present invention provides:

(1) A glutamine synthetase comprising amino acid sequence shown in SEQ ID NO: 1 in Sequence listing, wherein the tyrosine residue corresponding to the position 397 of SEQ ID NO: 1 is replaced with an amino acid residue other than tyrosine residue.

2) The glutamine synthetase according to (1), which comprises an amino acid sequence including deletion, substitution, insertion or addition of one or several amino acids at one or a plurality of positions other than the position 397 in the amino acid sequence shown in SEQ ID NO:1 in Sequence listing.

(3) The glutamine synthetase according to (1) or (2), wherein the residue corresponding to the position 397 of SEQ ID NO: 1 in Sequence listing is replaced with phenylalanine residue.

(4) The glutamine synthetase according to any of (1) to (3), wherein the glutamine synthetase is isolated from Escherichia coli.

(5) A DNA coding for the glutamine synthetase according to any of (1) to (4).

(6) The DNA according to claim 5, which is a DNA as defined in the following (a) or (b), wherein the codon of the tyrosine residue corresponding to the position 397 is replaced with a codon of amino acid other than tyrosine:

(a) a DNA which contains a nucleotide sequence of SEQ ID NO: 2 in Sequence Listing; or
(b) a DNA which is hybridizable with the nucleotide sequence of SEQ ID NO: 2 in Sequence Listing under the stringent conditions, the DNA coding for the protein which has glutamine synthetase activity and which is insensitive to indirect down-regulation by glutamine.
   (7) The DNA according to (6), wherein the stringent conditions is a condition in which washing is performed at 60° C., and at a salt concentration corresponding to 1×SSC and 0.1% SDS.
   (8) A bacterium, which is transformed with the DNA according to any of (5) to (7).
   (9) The bacterium according to (8), which belongs to the genus Escherichia.
   (10) The bacterium according to (8) or (9), which has an ability to produce L-amino acid.
   (11) A method for producing an L-amino acid, which method comprises the steps of: cultivating the bacterium according to any of (8) to (10) in a medium to produce and accumulate the L-amino acid in the medium, and collecting the L-amino acid from the medium.
   (12) The method according to (11), wherein the L-amino acid is selected from the group consisting of L-glutamine, L-arginine, L-tryptophan, L-histidine, L-glutamate.
   (13) The method according to (12), wherein the L-amino acid is L-glutamine.

The GS having a substitution at the tyrosine residue corresponding to the position 397 of SEQ ID NO: 1 in Sequence listing as described above may be referred to as “the mutant GS”, a DNA coding for the mutant GS may be referred to as “the mutant glnA gene”, and a GS without the substitution may be referred to as “a wild type GS”.

In the present specification, an amino acid is of L-configuration unless otherwise noted.

Further, the present invention will be explained in detail.

<1> Mutant GS and Mutant glnA Gene

It is known the tyrosine at position 397 is adenylylation site of GS (Numbers of amino acid residue of the enzyme are sited according to G Colombo and J J Villafranca (J. Biol. Chem., Vol. 261, Issue 23, 10587-10591, 1986). Adenylylation of GS leads to inactivation of the enzyme. The substitutions of the amino acid residue corresponding to the tyrosine at position 397 with any amino acid, preferably with phenylalanine, in the amino acid sequence of wild type GS leads to expression of a mutant protein with native level of activity and free from adenylylation. The mutant GS became insensitive to indirect down-regulation by glutamine.

The mutant GS can be obtained based on the sequences by introducing mutations into a wild type glnA gene using ordinary methods. As a wild type glnA gene, the glnA gene of E. coli can be mentioned (nucleotide numbers 6558 to 7967 in the sequence of GenBank Accession AE000462 U00096: SEQ ID NO: 2)

The mutant GS may include deletion, substitution, insertion, or addition of one or several amino acids at one or a plurality of positions other than 397, provided that the GS activity is not deteriorated. Term “GS activity” means activity to catalyze the reaction of formation the glutamine from glutamate and ammonia using ATP.

The number of “several” amino acids differs depending on the position or the type of amino acid residues in the three dimensional structure of the protein. This is because of the following reason. That is, some amino acids have high homology to one another and the difference in such an amino acid does not greatly affect the three dimensional structure of the protein. Therefore, the mutant GS of the present invention may be one which has homology of not less than 30 to 50%, preferably 50 to 70% with respect to the entire amino acid residues for constituting GS, and which has the GS activity.

In the present invention, “amino acid residue corresponding to the the position 397” means an amino acid sequence corresponding to the amino acid residue of position 397 in the amino acid sequence of SEQ ID NO: 1. A position of amino acid residue may change. For example, if an amino acid residue is inserted at N-terminus portion, the amino acid residue inherently locates at the position 397 becomes position 398. In such a case, the amino acid residue corresponding to the original position 397 is designated as the amino acid residue at the position 397 in the present invention.

The DNA, which codes for the substantially same protein as the mutant GS described above, may be obtained, for example, by modifying the nucleotide sequence, for example, by means of the site-directed mutagenesis method so that one or more amino acid residues at a specified site involve deletion, substitution, insertion, or addition. DNA modified as described above may be obtained by the conventionally known mutation treatment.

The substitution, deletion, insertion, or addition of nucleotide as described above also includes mutation, which naturally occurs (mutant or variant), for example, on the basis of the individual difference or the difference in species or genus of bacterium, which harbors GS.

The DNA coding for such variants can be obtained by isolating the DNA, which hybridizes with glnA gene or part of the gene under the stringent conditions, and which codes the protein having glutamine synthetase. The term “stringent conditions” referred to herein as a condition under which so-called specific hybrid is formed, and non-specific hybrid is not formed. For example, the stringent conditions includes a condition under which DNAs having high homology, for instance DNAs having homology no less than 70% to each other, are hybridized. Alternatively, the stringent conditions are exemplified by conditions which comprise ordinary condition of washing in Southern hybridization, e.g., 60° C., 1×SSC, 0.1% SDS, preferably 0.1×SSC, 0.1% SDS. As a probe for the DNA that codes for variants and hybridizes with glnA gene, a partial sequence of the nucleotide sequence of SEQ ID NO: 2 can also be used. Such a probe may be prepared by PCR using oligonucleotides produced based on the nucleotide sequence of SEQ ID NO: 2 as primers, and a DNA fragment containing the nucleotide sequence of SEQ ID NO: 2 as a template. When a DNA fragment in a length of about 300 bp is used as the probe, the conditions of washing for the hybridization consist of, for example, 50° C., 2×SSC, and 0.1% SDS.

<2> Bacterium Belonging to the Genus Escherichia of the Present Invention.

The bacterium belonging to the genus Escherichia of the present invention is a bacterium belonging to the genus Escherichia into which the mutant glnA gene described above is introduced. A bacterium belonging to the genus Escherichia is exemplified by E. coli. The mutant glnA gene can be introduced by, for example, transformation of a bacterium belonging to the genus Escherichia with a recombinant DNA comprising a vector which functions in a bacterium belonging to the genus Escherichia and the mutant glnA gene. The mutant glnA gene can be also introduced by substitution of glnA gene on a chromosome with the mutant glnA gene.

Vector using for introduction of the mutant glnA gene is exemplified by plasmid vectors such as pMW118, pBR322, pUC19 or the like, phage vectors such as 11059, 1BF101, M13mp9 or the like and transposon such as Mu, Tn10, Tn5 or the like.

The introduction of a DNA into a bacterium belonging to the genus Escherichia can be performed, for example, by a method of D. A. Morrison (Methods in Enzymology, 68, 326 (1979)) or a method in which recipient bacterial cell are treated with calcium chloride to increase permeability of DNA (Mandel, M., and Higa, A., J. Mol. Biol., 53, 159, (1970)) and the like.

A bacteria belonging to the genus Escherichia, which have an activity to produce significant amount of L-glutamine, are not known at present. It has been noticed that cultivation the E. coli strain K-12 in the nutrient medium, containing greater then 10 parts by weight of nitrogen per 100 parts by weight of carbon, leads to accumulation the 0.36 mg/ml of L-glutamine (Patent of Great Britain No. 1,113,117). So, a produced amount of L-glutamine can be increased by introduction of the mutant glnA gene into glutamic acid excreting wild-type bacterium belonging to the genus Escherichia.

As a L-glutamine-producing bacteria belonging to the other species are exemplified by Brevibacterium flavum FERM P-4272, Corynebacterium acetoacidophilum ATCC 13870, Microbacterium flavum FERM BP-664 (AJ 3684), Brevibacterium flavum FERM-BP 662 (AJ 3409), Corynebacterium acetoglutamicum ATCC 13870, Corynebacterium glutamicum FERM BP-663 (AJ 3682) (U.S. Pat. No. 5,164,307).

A produced amount of L-glutamine can be further increased by introduction of the mutant glnA gene into glutamic acid producing bacterium belonging to the genus Escherichia.

As the bacteria belonging to the genus Escherichia which have an activity to produce L-glutamic acid are exemplified by following E. coli strains: the strains, having resistance to an aspartic acid antimetabolite, and are deficient in alpha-ketoglutaric acid dehydrogenase activity, such as AJ13199 (FERM BP-5807) (U.S. Pat. No. 5,908,768), or strain FERM P-12379 additionally having low L-glutamic acid decomposing ability (U.S. Pat. No. 5,393,671); E. coli strain AJ13138 (FERM BP-5565) (U.S. Pat. No. 6,110,714) and the like.

As the bacteria belonging to the genus Escherichia which have an activity to produce L-arginine are exemplified by E. coli strain 237 (VKPM B-7925) (Russian Patent Application No. 2000116481), arginine producing strain into which argA gene encoding N-acetylglutamate synthetase is introduced (Japanese Laid-Open Publication No. 57-5693) and the like.

As the bacteria belonging to the genus Escherichia which have an activity to produce L-tryptophan are exemplified by E. coli strains which are derivatives of Genencor strain JB102/pBE7, carrying the tryptophan operon, the aroG gene and the serA gene from E. coli (U.S. Pat. No. 5,939,295), E. coli strains DSM10118, DSM10121, DSM10122, DSM10123 (U.S. Pat. No. 5,756,345), E. coli strain SV164(pGH5) (EP1149911A2), E. coli strains NRRL B-12257-NRRL B-12264 (U.S. Pat. No. 4,371,614) and the like.

As the bacteria belonging to the genus Escherichia which has an activity to produce L-histidine are exemplified by E. coli strain NRRL B-12116, NRRL B-12118, NRRL B-12119, NRRL B-12120, NRRL B-12121 (U.S. Pat. No. 4,388,405), and the like.

<3> Method for Producing L-Amino Acid.

The method of present invention includes method for producing L-amino acid, comprising steps of cultivating the bacterium of the present invention in a culture medium, to allow L-amino acid to be produced and accumulated in the culture medium, and collecting L-amino acid from the culture medium.

As explained in detail in the following Examples, the method of present invention includes method for producing L-glutamine, comprising steps of cultivating the bacterium of the present invention in a culture medium, to allow L-glutamine to be produced and accumulated in the culture medium, and collecting L-glutamine from the culture medium.

Glutamine donates nitrogen for the synthesis of purines and pyrimidines, and for some amino acids, such as L-arginine, L-tryptophan, L-histidine and L-glutamate. Glutamine plays significant role in L-arginine biosynthesis, since glutamine is used as the only physiological amino group donor for synthesis of carbamoylphosphate, which is a common precursor for L-arginine and the pyrimidines. In case of L-tryptophan formation, glutamine is utilized in the first reaction of tryptophan biosynthetic pathway, which involves the conversion of chorismate and glutamine to anthranilate, glutamate, and pyruvate. The nitrogen 3 of imidazole ring of L-histidine originates from glutamine. And finally, glutamine is used by glutamate oxoglutarate aminotransferase (GOGAT) in the synthesis of glutamate. When other pathways of biosynthesis of the abovementioned amino acids could be optimized for their overproduction, availability of glutamine might become one of the limiting factor. From the above, improving the ability to produce L-glutamine in a microorganism leads also to improved ability to produce L-arginine, L-tryptophan, L-histidine and L-glutamate in the microorganism. Therefore, the method of present invention includes method for producing L-arginine, comprising steps of cultivating the bacterium of the present invention in a culture medium, to allow L-arginine to be produced and accumulated in the culture medium, and collecting L-arginine from the culture medium. Also, the method of present invention includes method for producing L-tryptophan, comprising steps of cultivating the bacterium of the present invention in a culture medium, to allow L-tryptophan to be produced and accumulated in the culture medium, and collecting L-tryptophan from the culture medium. Also, the method of present invention includes method for producing L-histidine, comprising steps of cultivating the bacterium of the present invention in a culture medium, to allow L-histidine to be produced and accumulated in the culture medium, and collecting L-histidine from the culture medium. And, the method of present invention includes method for producing L-glutamate, comprising steps of cultivating the bacterium of the present invention in a culture medium, to allow L-glutamate to be produced and accumulated in the culture medium, and collecting L-glutamate from the culture medium.

In the method of present invention, the cultivation of the bacterium belonging to the genus Escherichia, the collection and purification of L-glutamine from the liquid medium may be performed in a manner similar to those of the conventional method for producing L-glutamine by fermentation using a bacterium. Also, in the method of present invention, the cultivation of the bacterium belonging to the genus Escherichia, the collection and purification of L-arginine from the liquid medium may be performed in a manner similar to those of the conventional method for producing L-arginine by fermentation using a bacterium. Also, in the method of present invention, the cultivation of the bacterium belonging to the genus Escherichia, the collection and purification of L-tryptophan from the liquid medium may be performed in a manner similar to those of the conventional method for producing L-tryptophan by fermentation using a bacterium. Also, in the method of present invention, the cultivation of the bacterium belonging to the genus Escherichia, the collection and purification of L-histidine from the liquid medium may be performed in a manner similar to those of the conventional method for producing L-histidine by fermentation using a bacterium. And, in the method of present invention, the cultivation of the bacterium belonging to the genus Escherichia, the collection and purification of L-glutamate from the liquid medium may be performed in a manner similar to those of the conventional method for producing L-glutamate by fermentation using a bacterium.

A medium used in cultivation may be either a synthetic medium or a natural medium, so long as the medium includes a carbon and a nitrogen source and minerals and, if necessary, nutrients the bacterium used requires for growth in appropriate amount. The carbon source may include various carbohydrates such as glucose and sucrose, and various organic acids, depending on assimilatory ability of the used bacterium. Alcohol including ethanol and glycerol may be used. As the nitrogen source, ammonia, various ammonium salts as ammonium sulfate, other nitrogen compounds such as amines, a natural nitrogen source such as peptone, soybean hydrolyzate and digested fermentative microbe are used. As minerals, monopotassium phosphate, magnesium sulfate, sodium chloride, ferrous sulfate, manganese sulfate, calcium carbonate are used. Some additional nutrient can be added to the medium if necessary. For instance, if the microorganism requires isoleucine for growth (isoleucine auxotrophy) the sufficient amount of isoleucine can be added to the medium for cultivation.

The cultivation is preferably the one under an aerobic condition such as a shaking, and aeration and stirring culture. The cultivation is usually performed at a temperature between 20 and 40° C., preferably 30 and 38° C. The cultivation is usually performed at a pH between 5 and 9, preferably between 6.5 and 7.2. The pH of the culture medium can be adjusted with ammonia, calcium carbonate, various acids, various bases, and buffers. Usually, a 1 to 3-day cultivation leads to the accumulation of the compounds in the medium.

The isolation of amino acid can be performed by removing solids such as cells from the medium by centrifugation or membrane filtration after cultivation, and then collecting and purifying such compounds by ion exchange, concentration and crystalline fraction methods and the like.

### BRIEF EXPLANATION OF THE DRAWINGS

FIG. 1 shows the relative position of primers SEQ ID NO: 3, 4 and 5.

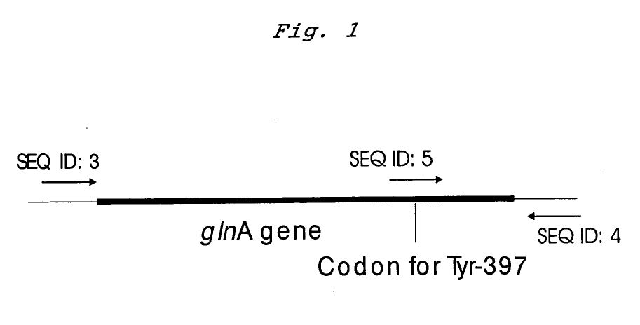

### BEST MODE FOR CARRYING OUT THE INVENTION

The present invention will be specifically explained with reference to the following examples.

### EXAMPLE 1

### Cloning the Mutant glnA Gene

The wild type glnA gene was obtained by amplification using PCR procedure and cloned into the vector pMW118 yielding the plasmid pMWglnA12. The chromosomal DNA of E. coli strain K12 was used as a template, oligonucleotides depicted in SEQ ID NO: 3 and 4 was used as primers. PCR procedure was carried out as follows: pretreatment at 94° C. for 5 min, then 40 cycles at 55° C. for 30 sec, 72° C. for 2 min, and 93° C. for 30 sec. Thus obtained PCR product was treated by XbaI and HindIII restrictases and ligated with the vector pMW118 plasmid previously treated with the same restrictases, yielding the plasmid pMWglnA12. To replace the TAT codon, encoding Tyr-397 in GS peptide, by the TTT codon, encoding phenylalanine, PCR procedure for site-directed mutagenesis was used. The pMWglnA12 plasmid carrying the wild type glnA gene was used as a template, oligonucleotides depicted in SEQ ID NO: 4 and 5 was used as primers. PCR procedure was carried out as follows: 55° C. for 30 sec, 72° C. for 1 min, and 94° C. for 30 sec, in 25 cycles. Thus obtained PCR product was treated by NcoI and HindIII restrictases and ligated with the plasmid pMWglnA12 plasmid previously treated with the same restrictases, yielding the plasmid pMWglnAphe-4.

### EXAMPLE 2

### Construction of an ilvA Deficient Derivative from the Wild Type Strain E. coil K12, having a Mutation in the ilvA Gene

The strain VL334 (VKPM B-1641) is an isoleucine auxotrophy and threonine auxotrophic strain, having mutations in thrC and ilva genes (U.S. Pat. No. 4,278,765). A wild type allele of thrC gene was transferred by the method of general transduction using bacteriophage P1, grown on cells of the wild type E. coli strain K12 (VKPM B-7). As a result, the isoleucine deficient strain VL334thrC was obtained.

Then, the plamid pMWglnAphe-4 was introduced into cells of the VL334thrC⁺ strain resulting the strain VL334thrC⁺/pMWglnAphe-4. As a control, the plasmid pMWglnA12 was also introduced into cells of the strain VL334thrC⁺ yielding the strain VL334thrC⁺/pMWglnA12.

### EXAMPLE 3

### Production of Glutamine and Glutamic Acid by the Strain Harboring the Mutant glnA Gene in Test-Tube Fermentation

The cultivation conditions in test-tube fermentation was as follows: the fermentation medium contained 60 g/l glucose, 35 g/l ammonia sulfate, 2 g/l KH₂PO₄, 1 g/l MgSO₄, 0.1 mg/l thiamine, 50 mg/l L-isoleucine, 5 g/l yeast extract Difco, 25 g/l chalk (pH 7.2). Glucose and chalk were sterilized separately. 2 ml of the medium was placed into test-tubes, inoculated with one loop of the tested microorganisms, and the cultivation was carried out at 37° C. for 2 days with shaking. The amount of produced glutamic acid and glutamine were determined by TLC (isopropanol:ethylacetate:ammonia:water=16:8 5:10 (v/v)). The results are shown in Table 1.

TABLE 1

| | Accumulation of | Accumulation |
|---|---|---|
| | glutamic acid, | of glutamine, |
| Strain | g/l | g/l |
| VL334thrC⁺ | 12.0 | 0 |
| VL334thrC⁺/pMWglnA12 | 7.5 | 0 |
| VL334thrC⁺/pMWglnAphe-4 | 1.3 | 1.3 |

As is shown in the table 1, the strain VL334thrC⁺/pMWglnAphe-4 carrying mutant glnA gene produced became able to produce L-glutamine.

1-13. (canceled)

14. A method for producing an L-amino acid, which method comprises:
cultivating a bacterium in a medium to produce and accumulate the L-amino acid in the medium, and collecting the L-amino acid from the medium, wherein said bacterium is a bacterium transformed with a DNA coding for a glutamine synthetase as defined in the following (a) or (b) whereby the codon of the tyrosine residue corresponding to the position 397 of SEQ ID NO: 1 is replaced with a codon of amino acid other than tyrosine:
(a) a DNA which contains a nucleotide sequence of SEQ ID NO: 2; or
(b) a DNA which is hybridizable with the nucleotide sequence of SEQ ID NO: 2 under the stringent conditions, which has homology not less than 70% to the nucleotide sequence of SEQ ID NO: 2, and codes for the protein which has glutamine synthetase activity.

15. The method according to claim 14, wherein said tyrosine residue corresponding to the position 397 of SEQ ID NO: 1 is replaced with phenylalanine residue.

16. The method according to claim 14, wherein said stringent conditions are conditions in which washing is performed at 60° C. and at a salt concentration corresponding to 1×SSC and 0.1% SDS.

17. A method for producing an L-amino acid, which method comprises:
cultivating a bacterium in a medium to produce and accumulate the L-amino acid in the medium, and collecting the L-amino acid from the medium, wherein said bacterium is a bacterium transformed with a DNA coding for a glutamine synthetase comprising amino acid sequence shown in SEQ ID NO: 1, whereby the tyrosine residue corresponding to the position 397 of SEQ ID NO: 1 is replaced with an amino acid residue other than tyrosine residue.

18. The method according to claim 17, wherein said tyrosine residue corresponding to the position 397 of SEQ ID NO: 1 is replaced with phenylalanine residue.

19. The method according to claim 14, wherein said bacterium belongs to the genus Escherichia.

20. The method according to claim 14, wherein the L-amino acid is selected from the group consisting of L-glutamine, L-arginine, L-tryptophan, L-histidine and L-glutamate.

21. The method according to claim 14, wherein the L-amino acid is L-glutamine.
